(19) United States
(12) Patent Application Publication
(10) Pub. No.: US 2021/0335500 A1
(43) Pub. Date: Oct. 28, 2021

(54) METHOD AND DEVICE FOR PREDICTING A NUMBER OF CONFIRMED CASES OF AN INFECTIOUS DISEASE, APPARATUS, AND STORAGE MEDIUM

(71) Applicant: SOUTH UNIVERSITY OF SCIENCE AND TECHNOLOGY OF CHINA, Shenzhen (CN)

(72) Inventors: Xuan Song, Shenzhen (CN); Haoran Zhang, Shenzhen (CN); Liqiao Huang, Shenzhen (CN); Ryosuke Shibasaki, Shenzhen (CN)

(21) Appl. No.: 16/928,762

(22) Filed: Jul. 14, 2020

(30) Foreign Application Priority Data
Apr. 24, 2020 (CN) 202010330942.1

(51) Int. Cl. G16H 50/80; G06F 17/18; G06F 17/13; G06F 16/951

(52) U.S. Cl. CPC G16H 50/80; G06F 16/951; G06F 17/13; G06F 17/18

(57) ABSTRACT

A method and device for predicting a number of confirmed cases of an infectious disease, an apparatus, and a storage medium. The method includes obtaining a number of historical confirmed cases corresponding to each of multiple historical time periods adjacent to a time period to be predicted, adding a number of historical confirmed cases corresponding to a current historical time period with numbers of historical confirmed cases corresponding to previous target historical time periods to determine a historical cumulative number corresponding to each current historical time period, determining a predicted cumulative number corresponding to the time period to be predicted based on the historical cumulative number corresponding to each current historical time period, and determining a differential value between the predicted cumulative number corresponding to the time period to be predicted and a historical cumulative number corresponding to current target historical time period adjacent to the time period to be predicted, and taking the differential value as a predicted confirmed number of the time period to be predicted.

### CROSS-REFERENCE TO RELATED APPLICATION

This application claims priority to and benefit of China Patent Application No. 202010330942.1 filed on Apr. 24, 2020, the disclosure of which is hereby incorporated herein by reference in its entirety.

### TECHNICAL FIELD

Embodiments of the present disclosure relate to the technical field of infection prediction, and more particularly relate to a method and device for predicting a number of confirmed cases of an infectious disease, an apparatus, and a storage medium.

### BACKGROUND

With the increasing occurrence of infectious diseases, the scientific community and all sectors of the society have carried out prediction research on the development of epidemic situation. It is of great and urgent significance to predict the future development trend of infectious diseases.

Currently, in predicting the number of confirmed cases with novel coronavirus pneumonia, the historical data is based on to determine the daily increased number of confirmed cases, which is further used to predict the future predicted increased number, thereby determining the number of confirmed cases in the future.

However, there will be fluctuations in the daily increased number of confirmed cases, resulting in inaccurate projections of the number of confirmed cases in the future.

### SUMMARY

Embodiments of the present disclosure provide a method and device for predicting a number of confirmed cases of an infectious disease, an apparatus and a storage medium to improve the accuracy of predicting the number of future confirmed cases.

In a first aspect, embodiments of the present disclosure provide a method for predicting a number of confirmed cases of an infectious disease, the method including the following:
obtaining a number of historical confirmed cases corresponding to each of a plurality of historical time periods adjacent to a time period to be predicted;
adding a number of historical confirmed cases corresponding to a current historical time period with numbers of historical confirmed cases corresponding to previous target historical time periods, to determine a historical cumulative number corresponding to each current historical time period;
determining a predicted cumulative number corresponding to the time period to be predicted based on the historical cumulative number corresponding to each current historical time period; and
determining a differential value between the predicted cumulative number corresponding to the time period to be predicted and the historical cumulative number corresponding to the current target historical time period adjacent to the time period to be predicted, and taking the differential value as a predicted confirmed number of the time period to be predicted.

Optionally, the operation of determining the predicted cumulative number corresponding to the time period to be predicted based on the historical cumulative number corresponding to each current historical time period may include the following:
performing computation on the historical cumulative number corresponding to each current historical time period using a particle swarm optimization (PSO) algorithm, to determine a target dynamic weight coefficient corresponding to each current historical time period;
determining a development coefficient a and a greyscale driving coefficient b based on the historical cumulative number corresponding to each current historical time period; and
determining the predicted cumulative number corresponding to the time period to be predicted based on a first preset formula, is the first preset formula being:

$${\hat{x}}^{( 1 )} ( k ) = [ {\sum }_{i = 1}^{n} {w}^{n - i} {x}^{( 1 )} ( i ) - \frac{b}{a} ] {e}^{- a ( k - t )} + \frac{b}{a} ,$$

where w^{n-k} denotes the target dynamic weight coefficient, \hat{x}⁽¹⁾(k) denotes the predicted cumulative number corresponding to the time period to be predicted, and x⁽¹⁾(i) denotes the historical cumulative number corresponding to the current historical time period.

Optionally, the operation of performing computation on the historical cumulative number corresponding to each current historical time period using the PSO algorithm, and determining the target dynamic weight coefficient corresponding to each current historical time period includes steps described below.
performing computation on the historical cumulative number corresponding to each current historical time period using the PSO algorithm to obtain a two-dimensional vector [w,t]^{T}; and
extracting an initial dynamic weight coefficient from the two-dimensional vector, and using the initial dynamic weight coefficient as the target dynamic weight coefficient.

Optionally, the operation of performing computation on the historical cumulative number corresponding to each current historical time period using the PSO algorithm, and determining the target dynamic weight coefficient corresponding to each current historical time period includes steps described below.
performing computation on the historical cumulative number corresponding to each current historical time period using the PSO algorithm to obtain a two-dimensional vector [w,t]^{T};
extracting an initial dynamic weight coefficient from the two-dimensional vector;
introducing the initial dynamic weight coefficient as a constant into McLaughlin series; and
performing computation on the McLaughlin series using an ant colony optimization algorithm (ACO) algorithm to obtain a final dynamic weight coefficient, and using the final dynamic weight coefficient as the target dynamic weight coefficient.

Optionally, the operation of performing computation on the McLaughlin series using the ACO algorithm to obtain the final dynamic weight coefficient, and using the final dynamic weight coefficient as the target dynamic weight coefficient includes steps described below.
calculating an intermediate dynamic weight parameter using the ACO algorithm, wherein the number of calculation times is incremented by one with each calculation of the intermediate dynamic weight parameter;
determining whether the number of current calculation times reaches a preset number of times; and
taking the corresponding intermediate dynamic weight parameter when the number of current calculation times reaches the preset number of times as the target dynamic weight coefficient.

Optionally, the operation of determining the development coefficient a and the greyscale driving coefficient b based on the historical cumulative number corresponding to each current historical time period may include the following:
determining a reference number corresponding to each current historical time period based on the target dynamic weight coefficient corresponding to each current historical time period and the historical cumulative number corresponding to the current historical time period;
accumulating the product of the reference number corresponding to each current historical time period with a preset coefficient and substituting the result into a grey differential equation, wherein the grey differential equation comprises a correspondence relation between the greyscale driving coefficient b and the development coefficient a; and
solving the grey differential equation by a least square method to obtain the development coefficient a and the greyscale driving coefficient b.

Optionally, the operation of obtaining the number of historical confirmed cases corresponding to each of the plurality of historical time periods adjacent to the time period to be predicted may include the following:
crawling a website to obtain the number of historical confirmed cases corresponding to each of the plurality of historical time periods adjacent to the time period to be predicted.

In a second aspect, embodiments of the present disclosure provide a device for predicting a number of confirmed cases of an infectious disease, the device including a historical confirmed number determination module, a historical cumulative number determination module, a predicted cumulative number determination module and a predicted confirmed number determination module.

The historical confirmed number determination module is configured for obtaining a number of historical confirmed cases corresponding to each of a plurality of historical time periods adjacent to a time period to be predicted.

The historical cumulative number determination module is configured for adding a number of historical confirmed cases corresponding to a current historical time period with numbers of historical confirmed cases corresponding to previous target historical time periods, to determine a historical cumulative number corresponding to each current historical time period.

The predicted cumulative number determination module is configured for determining a predicted cumulative number corresponding to the time period to be predicted based on the historical cumulative number corresponding to each current historical time period.

The predicted confirmed number determination module is configured for determining a differential value between the predicted cumulative number corresponding to the time period to be predicted and a historical cumulative number corresponding to current target historical time period adjacent to the time period to be predicted, and taking the differential value as a predicted confirmed number of the time period to be predicted.

In a third aspect, an embodiment of the present disclosure provides an apparatus, which includes one or more processors and a storage device.

The storage device stores one or more computer programs.

When the one or more computer programs are executed by the one or more processors, the one or more processors are caused to perform the method for predicting a number of confirmed cases of an infectious disease of any one of embodiments of the present disclosure.

In a fourth aspect, an embodiment of the present disclosure provides a computer-readable storage medium storing a computer program. When executed by a processor, the computer program causes the method for predicting a number of confirmed cases of an infectious disease of any one of embodiments of the present disclosure to be performed.

In the embodiments of the present disclosure, the number of historical confirmed cases corresponding to each of the plurality of historical time periods adjacent to the time period to be predicted is obtained; the historical cumulative number corresponding to each current historical time period is then determined by adding the number of historical confirmed cases corresponding to the current historical time period with the number of historical confirmed cases corresponding to the previous target historical time period; the predicted cumulative number corresponding to the time period to be predicted is determined based on the historical cumulative number corresponding to each current historical time period; and the differential value between the predicted cumulative number corresponding to the time period to be predicted and the historical cumulative number corresponding to current target historical time period adjacent to the time period to be predicted is determined, and the differential value is taken as the predicted confirmed number of the time period to be predicted. Thus, the problem of inaccurate prediction of the number of future confirmed cases is solved, thereby improving the accuracy of predicting the number of future confirmed cases.

### BRIEF DESCRIPTION OF DRAWINGS

FIG. 1 is a flowchart of a method for predicting a number of confirmed cases of an infectious disease according to Embodiment one of the present disclosure.

FIG. 2 is a flowchart of a method for predicting a number of confirmed cases of an infectious disease according to Embodiment two of the present disclosure.

FIG. 3 is a block diagram of a device for predicting a number of confirmed cases of an infectious disease according to Embodiment three of the present disclosure.

FIG. 4 is a block diagram of an apparatus according to Embodiment four of the present disclosure.

### DETAILED DESCRIPTION

Hereinafter the present disclosure will be further described in detail in conjunction with the drawings and embodiments. It is to be understood that the specific embodiments set forth herein are merely intended to illustrate rather than limiting the present disclosure. Additionally, it is to be noted that for ease of description, merely part, instead of all, of the structures related to the present disclosure are illustrated in the drawings.

Before discussing exemplary embodiments in more detail, it is to be mentioned that some exemplary embodiments are described as processes or methods depicted as flowcharts. Although the flowchart describes the steps as sequential processing, many of them may be implemented concurrently, concomitantly or simultaneously. In addition, an order of the steps may be rearranged. The processing may be terminated when an operation is completed, but may also have additional steps not included in the drawings. The processing may correspond to methods, functions, procedures, subroutines, subprograms and the like.

In addition, terms “first”, “second” and the like may be used herein to describe various directions, actions, steps or elements, but these directions, actions, steps or elements are not limited by these terms. These terms are only used to distinguish a first direction, action, step or element from another direction, action, step or element. For example, without departing from the scope of the present application, first information may be referred to as second information, and similarly, the second information may be referred to as the first information. Both the first information and the second information are information but are not same information. The terms “first,” “second,” and the like cannot be understood as indicating or implying relative importance or implicitly illustrating a number of indicated technical features. Therefore features defined as “first” and “second” may explicitly or implicitly include one or more the features. In the description of the present disclosure, “multiple” means at least two, for example, two, three, or the like, unless otherwise expressly defined.

### Embodiment One

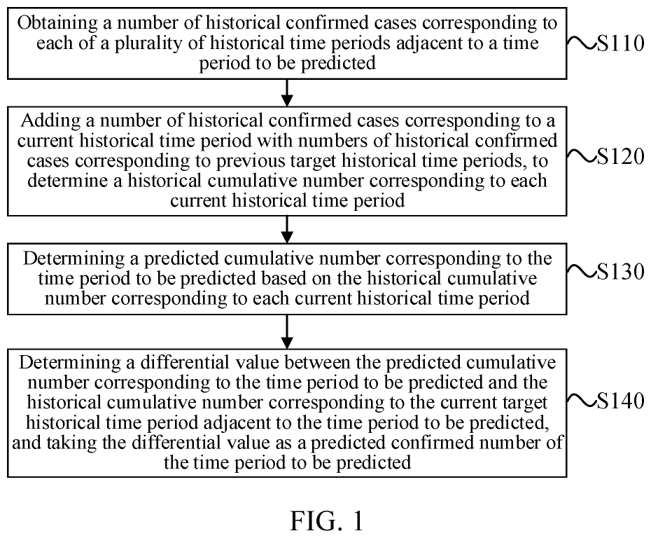

FIG. 1 is a flowchart of a method for predicting a number of confirmed cases of an infectious disease according to Embodiment one of the present disclosure and may be suitable for scenarios of predicting the number of future confirmed cases of infectious diseases. The method may be implemented by a device for predicting a number of confirmed cases of an infectious disease, and the device may be implemented in software and/or hardware and may be integrated on an apparatus.

As illustrated in FIG. 1, the method for predicting the number of confirmed cases of the infectious disease provided by Embodiment one of the present disclosure includes steps described below.

In S110, the number of historical confirmed cases corresponding to each of a plurality of historical time periods adjacent to a time period to be predicted is obtained.

The time period to be predicted may be in units of hours, days, months, and the like, which is no limited here. The historical time period is adjacent time period before the time period to be predicted, and the historical time period has a same unit as the time period to be predicted. Exemplarily, when the time period to be predicted is 11th, the plurality of historical time periods may be 10th, 9th, 8th and the like. The number of historical confirmed cases refers to a total confirmed number in the historical time period, and it may be a number of historical confirmed cases with a time period node at 12:00 p.m. in the historical time period. Optionally, the number of historical confirmed cases may be input into the device for predicting the number of confirmed cases of the infectious disease after queried manually, or may further be the number of historical confirmed cases corresponding to each of the plurality of historical time periods adjacent to the time period to be predicted crawled from a web page, which is not specifically limited here.

In S10, a historical cumulative number corresponding to each current historical time period is determined by adding a number of historical confirmed cases corresponding to a current historical time period with numbers of historical confirmed cases corresponding to previous target historical time periods.

Each historical time period may serve as current historical time period, and number of historical confirmed cases corresponding to a current historical time period and the number of historical confirmed cases corresponding to the target historical time period before the current historical time period are summed up to obtain the historical cumulative number corresponding to each current historical time period. The target historical time period is all historical time period before the current historical time period in the plurality of historical time periods. Exemplarily, the plurality of historical time periods is 10th, 9th, and 8th, the number of confirmed cases of 8th is 10, the number of confirmed cases of 9th is 30, and the number of confirmed cases of 10th is 50, then the historical cumulative number of 8th is 10, the historical cumulative number of 9th is 10+30=40, and the historical cumulative number of 10th is 10+30+50=90.

In S130, a predicted cumulative number corresponding to the time period to be predicted is determined based on the historical cumulative number corresponding to each current historical time period.

The predicted cumulative number is a sum of a predicted confirmed number of the time period to be predicted and the number of historical confirmed cases corresponding to each of the plurality of historical time periods, and may also be understood as a sum of the predicted confirmed number and a historical cumulative number corresponding to current target historical time period adjacent to the time period to be predicted. In this embodiment, there is no limitation on how to determine the predicted cumulative number of the time period to be predicted based on the historical cumulative number corresponding to each current historical time period, for example, it may be determined by curve fitting or a grey model.

In S140, a differential value between the predicted cumulative number corresponding to the time period to be predicted and the historical cumulative number corresponding to the current target historical time period adjacent to the time period to be predicted is determined, and the differential value is taken as the predicted confirmed number of the time period to be predicted.

The current target historical time period is current historical time period closest to the time period to be predicted. Exemplarily, the time period to be predicted is 11th, the predicted cumulative number of 11th is 200, 10th is the current target historical time period of the time period to be predicted, and the historical cumulative number of 10th is 90, then the differential value is 110, that is, the predicted confirmed number of 11th is 110.

In this embodiment, specifically, a prediction is performed by the historical cumulative number corresponding to the current historical time period, and a rule of continuous growth is obtained. Therefore, the predicted cumulative number is determined first, thereby obtaining the predicted confirmed number of the time period to be predicted. In the existing prediction based on the increased number, the prediction result may be inaccurate when there is fluctuation in the increased number. Compared with the existing prediction, this embodiment derives the rule of continuous growth by predicting based on the historical cumulative number, thereby ensuring the accuracy of prediction even if there is fluctuation.

In the technical solution of the embodiments of the present disclosure, the number of historical confirmed cases corresponding to each of the plurality of historical time periods adjacent to the time period to be predicted is obtained; the historical cumulative number corresponding to each current historical time period is determined by summing up the number of historical confirmed cases corresponding to the current historical time period with the numbers of historical confirmed cases corresponding to the previous target historical time periods; the predicted cumulative number corresponding to the time period to be predicted is determined based on the historical cumulative number corresponding to each current historical time period; and the differential value between the predicted cumulative number corresponding to the time period to be predicted and the historical cumulative number corresponding to current target historical time period adjacent to the time period to be predicted is determined, and the differential value is taken as the predicted confirmed number of the time period to be predicted. In the existing prediction based on the increased number, the prediction result may be inaccurate when there is fluctuation in the increased number. Compared with the existing prediction, this embodiment derives the rule of continuous growth by predicting based on the historical cumulative number, thereby ensuring the accuracy of the prediction even if there is fluctuation, improving the accuracy of predicting the number of future confirmed cases.

### Embodiment Two

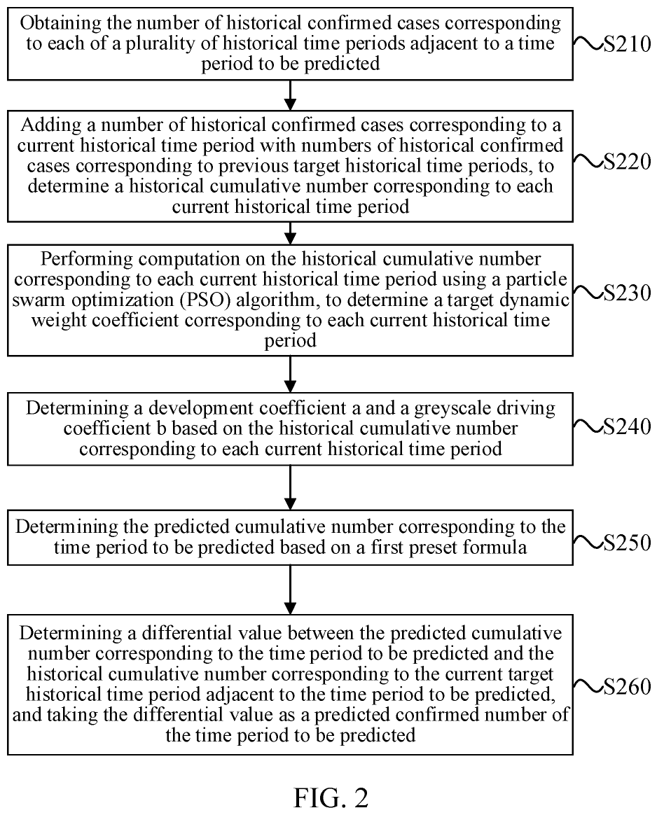

FIG. 2 is a flowchart of a method for predicting a number of confirmed cases of an infectious disease according to Embodiment two of the present disclosure. This embodiment is a further refinement of the above-mentioned technical scheme, and a further refinement is carried out to determine a predicted cumulative number corresponding to a time period to be predicted according to historical cumulative number corresponding to each current historical time period. The method may be implemented by a device for predicting a number of confirmed cases of an infectious disease, and the device may be implemented in software and/or hardware and may be integrated on an apparatus.

As illustrated in FIG. 2, the method for predicting the number of confirmed cases of the infectious disease provided by Embodiment two of the present disclosure may include the following operations.

In S210, a number of historical confirmed cases corresponding to each of a plurality of historical time periods adjacent to a time period to be predicted is obtained.

In S220, a historical cumulative number corresponding to each current historical time period is determined according to a sum of a number of historical confirmed cases corresponding to a current historical time period and numbers of historical confirmed cases corresponding to previous target historical time periods.

In S230, computation is performed on the historical cumulative number corresponding to each current historical time period using a particle swarm optimization (PSO) algorithm, thereby determining a target dynamic weight coefficient corresponding to each current historical time period.

A first step of the particle swarm optimization algorithm is to initialize a population size of particle swarm and generate first generation particles. Then, each particle calculates its fitness value by using a relative error between a predicted value of the historical time period and the historical cumulative number as a fitness value. In each iteration time period, an optimal individual value and group value are found and updated. The target dynamic weight coefficient corresponding to each current historical time period may be determined using the PSO algorithm.

In an optional implementation mode, the step of performing computation on the historical cumulative number corresponding to each current historical time period using the PSO algorithm, and determining the target dynamic weight coefficient corresponding to each current historical time period may include the following operations

Computation is performed on the historical cumulative number corresponding to each current historical time period using the PSO algorithm, to obtain a two-dimensional vector [w,t]^{T}. An initial dynamic weight coefficient is extracted from the two-dimensional vector, and the initial dynamic weight coefficient is taken as the target dynamic weight coefficient.

In this implementation, specifically, the particle swarm optimization algorithm assumes that there is a group of random particles, and then speeds and positions of the group of random particles are initialized in a certain interval and search space.

xᵢ=rand×xₘₐₓ,i=1

vᵢ=rand×vₘₐₓ,i=1

xₘₐₓ denotes a maximum value of a particle position, vₘₐₓ denotes an upper limit of a particle velocity, and xₘₐₓ and xₘₐₓ may be estimated within a certain range.

The position and velocity of each particle are updated according to the following formula:

xᵢ=xᵢ+vᵢ,i=2,3, . . .

vᵢ=w×vᵢ+c_{1×r}and×(pbestᵢ−xᵢ)+c₂×rand×(gbestᵢ−xᵢ),i=2,3, . . .
c₁ and c₂ are acceleration coefficients, and randϵ[0,1].

An optimal result is obtained by iterations. Each particle updates its position by the following two optimal values: one is determined by each particle and expressed as an individual best (recorded as pbest), and the other is expressed as an entire population (recorded as gbest). Each particle updates its value when an individual fitness value conforms to a comparison expression pbest(i)<gbest.

An evaluation function of the fitness value in the particle swarm optimization algorithm adopts a minimum average absolute percentage error, which is constructed as follows:

$$\begin{matrix}{fitness [ Q ( i , j ) ]}^{T} = \frac{1}{n } {\sum }_{k = 1}^{n} \frac{\vert {\hat{x}}^{( 0 )} ( k ) - {x}^{( 0 )} ( k ) \vert}{{x}^{( 0 )} ( k )} \\ = \frac{1}{n } {\sum }_{k = 1}^{n} \frac{\vert [ {\sum }_{i = 1}^{n} {w}^{n - i} {x}^{( 1 )} ( i ) - \frac{b}{a} ] ( 1 - {e}^{a} ) {e}^{- a ( k - t )} - {x}^{( 0 )} ( k ) \vert}{{x}^{( 0 )} ( k )} .\end{matrix}$$

In this embodiment, the position of each particle is expressed as a vector [w,t]^{T}, and the vector [w,t]^{T} is a two-dimensional vector. A specific calculation method follows the above-mentioned rules. In this implementation, the initial dynamic weight coefficient w of the two-dimensional vector [w,t]^{T} is taken as the target dynamic weight coefficient.

In another optional implementation, the operation of performing computation on the historical cumulative number corresponding to each current historical time period using the PSO algorithm, and determining the target dynamic weight coefficient corresponding to each current historical time period may include the following operations.

Computation is performed on the historical cumulative number corresponding to each current historical time period using the PSO algorithm to obtain the two-dimensional vector [w,t]^{T}. The initial dynamic weight coefficient is extracted from the two-dimensional vector. The initial dynamic weight coefficient is introduced as a constant into McLaughlin series. Then computation is performed on the McLaughlin series using an ant colony optimization algorithm (ACO) algorithm to obtain a final dynamic weight coefficient, and the final dynamic weight coefficient is taken as the target dynamic weight coefficient.

In this implementation, the initial dynamic weight coefficient is further optimized to obtain the final dynamic weight coefficient, such that the final dynamic weight coefficient is taken as the target dynamic weight coefficient. Specifically, the initial dynamic weight coefficient w is substituted into the McLaughlin series and replaces ƒ(x)=w^{x-1}, where the initial dynamic weight parameter w is a constant and x is a variable, and an expression is obtained.

$$f ( x ) = {w}^{x - 1} = {w}^{x - 1} {{\vert}_{x = 0} + ( {w}^{x - 1} ln w ) \vert}_{x = 0} + \frac{( {w}^{x - 1} {ln}^{2} w ) {\vert}_{x = 0}}{2 !} {x}^{2} + \frac{( {w}^{x - 1} {ln}^{3} w ) {\vert}_{x = 0}}{3 !} {x}^{3} + … + \frac{{( {w}^{x - 1} {ln}^{n} w )}_{x = 0}}{n !} {x}^{n} + \frac{{( {w}^{x - 1} {ln}^{n + 1} w ) \vert}_{x = 0}}{( n + 1 ) !} {x}^{n + 1} .$$

When the McLaughlin series is calculated using the ACO algorithm, relevant parameters need to be initialized, an ant colony size is included and recorded as numberofant, a maximum iteration number is recorded as iterationnumber, a maximum step limit is steplimitation, and an initial iteration number is iter=1. An initial position of the ant colony initialposition=(ƒ(1), ƒ(2), ƒ(3), . . . , ƒ(k)) is obtained by McLaughlin expansion.

During each iteration, the position of each individual in the ant colony is recorded as a two-dimensional variable antposition with a same number of rows as numberofant and a same number of columns as initialposition. An optimal position of the whole colony in history is referred to as historypositionbest. A calculation process of each particle position is expressed as process defined as a matrix with a same dimension as antposition and depicting specific steps of estimating particle paths.

A number of iterations is updated from 1 to iterationnumber. The historical optimal position takes a position of a minimum fitness value in a previous iteration.

historypositionbest(i+1,:)=historypositionbest(i,:)

An optimal position for each individual is updated as follows:

antposition(j,:)=historypositionbest(i,:)

The position of the particle in each iteration is changed and a movement step size of the particle is limited. A random motion in each direction is iterated by the following equation:

antposition(j,:)=antposition(j−1,:)+

(step lim itation(1,2)−step lim itation(1,1))*(rand−0.5);

A distance of each ant path is calculated, and an optimal solution (a shortest path) is recorded in a current number of iterations. At the same time period, a pheromone concentration on a connection path of each location is updated. After the iteration is completed, the number of iterations is calculated, historyposition(nn,:)=antposition(j,:) is the fitness value obtained by the process, and if m<minvalue,

min value=m,process(i+1,1)=minvalue

historypositionbest(i+1,:)=antposition(j,:)

A value of antposition of the jth iteration is assumed the smallest. xstar is defined as the optimal result of the algorithm. By adjusting the initial dynamic weight coefficient before a high-order polynomial, an error can be minimized, thus obtaining a more accurate fitting equation and improving the prediction accuracy. By replacing coefficients in the McLaughlin formula expansion with a newly obtained xstar, a more accurate [w,t]^{T} result can be obtained.

Optionally, a calculation function of the fitness value in the ant colony algorithm also adopts the minimum average absolute percentage error which is the same as the particle swarm optimization algorithm, and its expression is as follows:

$$\begin{matrix}process ( i , \text{:} ) = {fitness [ Q ( i , j ) ]}^{T} = \frac{1}{n} {\sum }_{k = 1}^{n} \frac{\vert {\hat{x}}^{( 0 )} ( k ) - {x}^{( 0 )} ( k ) \vert}{{x}^{( 0 )} ( k )} \\ = \frac{1}{n} {\sum }_{k = 1}^{n} \frac{\vert [ {\sum }_{i = 1}^{n} {w}^{n - i} {x}^{( 1 )} ( i ) - \frac{b}{a} ] ( 1 - {e}^{a} ) {e}^{- a ( k - t )} - {x}^{( 0 )} ( k ) \vert}{{x}^{( 0 )} ( k )} .\end{matrix}$$

In an optional implementation mode, the step of performing computation on the McLaughlin series using the ACO algorithm to obtain the final dynamic weight coefficient, and taking the final dynamic weight coefficient as the target dynamic weight coefficient may include the following operations.

An intermediate dynamic weight parameter is calculated using the ACO algorithm, where the number of calculation times is incremented by one with each calculation of the intermediate dynamic weight parameter. Determination is made as to the number of current calculation times reaches a preset number of times. An intermediate dynamic weight parameter when the number of current calculation times reaches the preset number of times is taken as the target dynamic weight coefficient.

In this implementation, it can be expressed as follows:

xstar=historypositionbest(iterationnumaber+1,:)

When the number of current calculation times is less than the preset number of times, the number of iterations is incremented by 1 and the step of constructing a solution space is returned; otherwise, the iteration ends and the optimal solution xstar is output.

In S240, a development coefficient a and a greyscale driving coefficient b are determined based on the historical cumulative number corresponding to each current historical time period.

The development coefficient a and the greyscale driving coefficient b refer to parameters used to determine the predicted cumulative number, which can be determined by the historical cumulative number.

In an optional implementation, the operation of determining the development coefficient a and the greyscale driving coefficient b based on the historical cumulative number corresponding to each current historical time period may include the following.

A reference number corresponding to each current historical time period is determined according to the target dynamic weight coefficient corresponding to each current historical time period and the historical cumulative number corresponding to each current historical time period. A product of the reference number corresponding to each current historical time period with a preset coefficient is accumulated and the result is substituted into a grey differential equation, where the grey differential equation includes a correspondence relation between the greyscale driving coefficient b and the development coefficient a. The grey differential equation is solved by a least square method to obtain the development coefficient a and the greyscale driving coefficient b.

In this implementation, the reference number corresponding to each current historical time period may be referred to as w^{n-k}x⁽¹⁾(k), and n denotes a total number of the plurality of historical time periods. The product of the reference number corresponding to each current historical time period and the preset coefficient is accumulated, and z⁽¹⁾(k)=0.5x⁽¹⁾(k−1)+0.5x⁽¹⁾(k), k=2, 3, . . . , n. The grey differential equation is:

x⁽⁰⁾(k)+az⁽¹⁾(k)=b. A winterization equation is

$$\frac{d {x}^{( 1 )}}{d t} + a {x}^{( 1 )} = b .$$

The development coefficient a and the greyscale driving coefficient b may be obtained by solving with the least square method. Specifically,

$$[ \begin{matrix}a \\ b\end{matrix} ] = {( {B}^{T} B )}^{- 1} {B}^{T} Y , \qquad ( 3 )$$

where

$$B = [ \begin{matrix}- {z}^{( 1 )} ( 2 ) & 1 \\ - {z}^{( 1 )} ( 3 ) & 1 \\ ⋮ & ⋮ \\ - {z}^{( 1 )} ( n ) & 1\end{matrix} ] , Y = [ \begin{matrix}{x}^{( 0 )} ( 2 ) \\ {x}^{( 0 )} ( 3 ) \\ ⋮ \\ {x}^{( 0 )} ( n )\end{matrix} ] .$$

Therefore, calculation results of a and b may be obtained.

In S250, the predicted cumulative number corresponding to the time period to be predicted is determined based on a first preset formula, is the first preset formula being:

$${\hat{x}}^{( 1 )} ( k ) = [ {\sum }_{i = 1}^{n} {w}^{n - i} {x}^{( 1 )} ( i ) - \frac{b}{a} ] {e}^{- a ( k - t )} + \frac{b}{a} ;$$

where w^{n-k} denotes the target dynamic weight coefficient, \hat{n}⁽¹⁾(k) denotes the predicted cumulative number corresponding to the time period to be predicted, and x⁽¹⁾(i) denotes a historical cumulative number corresponding to the current historical time period.

In this step, the development coefficient a, the greyscale driving coefficient b, the target dynamic weight coefficient and the historical cumulative number corresponding to the current historical time period have been determined through previous steps, so the predicted cumulative number of the time period to be predicted may be directly solved by directly substituted into the first preset formula.

In S260, a differential value between the predicted cumulative number corresponding to the time period to be predicted and a historical cumulative number corresponding to current target historical time period adjacent to the time period to be predicted is determined, and the differential value is taken as a predicted confirmed number of the time period to be predicted.

In the technical solution of the embodiments of the present disclosure, the number of historical confirmed cases corresponding to each of the plurality of historical time periods adjacent to the time period to be predicted is acquired; the historical cumulative number corresponding to each current historical time period is determined according to the sum of the number of historical confirmed cases corresponding to the current historical time period and the number of historical confirmed cases corresponding to the previous target historical time period; the predicted cumulative number corresponding to the time period to be predicted is determined based on the historical cumulative number corresponding to each current historical time period; and the differential value between the predicted cumulative number corresponding to the time period to be predicted and the historical cumulative number corresponding to current target historical time period adjacent to the time period to be predicted is determined, and the differential value is taken as the predicted confirmed number of the time period to be predicted. In the existing prediction which is based on the increased number, the prediction result may be inaccurate when there is fluctuation in the increased number. Compared with the existing prediction, this embodiment derives the rule of continuous growth by predicting based on the historical cumulative number, thereby ensuring the accuracy of prediction even if there is fluctuation, improving the accuracy of predicting the number of future confirmed cases.

### Embodiment Three

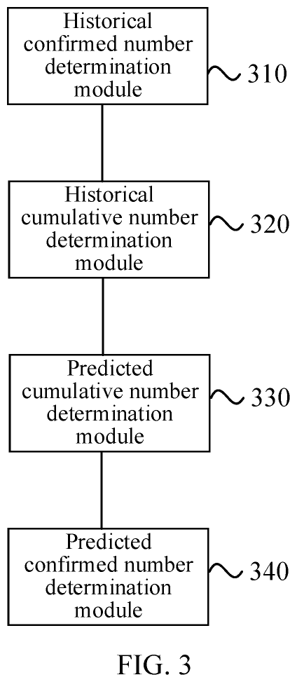

FIG. 3 is a block diagram of a device for predicting a number of confirmed cases of an infectious disease according to Embodiment three of the present disclosure. This embodiment is suitable for scenarios of predicting the number of future confirmed cases of infectious diseases. The device may be implemented in software and/or hardware and may be integrated on an apparatus.

As illustrated in FIG. 3, the device for predicting the number of confirmed cases of the infectious disease provided by this embodiment may include a historical confirmed number determination module 310, a historical cumulative number determination module 320, a predicted cumulative number determination module 330 and a predicted confirmed number determination module 340.

The historical confirmed number determination module 310 is used for acquiring a number of historical confirmed cases corresponding to each of a plurality of historical time periods adjacent to a time period to be predicted. The historical cumulative number determination module 320 is used for determining a historical cumulative number corresponding to each current historical time period according to a sum of a number of historical confirmed cases corresponding to a current historical time period and numbers of historical confirmed cases corresponding to previous target historical time periods. The predicted cumulative number determination module 330 is used for determining a predicted cumulative number corresponding to the time period to be predicted based on the historical cumulative number corresponding to each current historical time period. The predicted confirmed number determination module 340 is used for determining a differential value between the predicted cumulative number corresponding to the time period to be predicted and a historical cumulative number corresponding to current target historical time period adjacent to the time period to be predicted, and taking the differential value as a predicted confirmed number of the time period to be predicted.

Optionally, the predicted cumulative number determination module 330 includes a coefficient determination unit and a predicted cumulative number determination unit. The coefficient determination unit is used for determining a target dynamic weight coefficient corresponding to each current historical time period by calculating the historical cumulative number corresponding to each current historical time period using a particle swarm optimization (PSO) algorithm; and determining a development coefficient a and a greyscale driving coefficient b based on the historical cumulative number corresponding to each current historical time period. The predicted cumulative number determination unit is used for determining a predicted cumulative number of the time period to be predicted based on a first preset formula, is the first preset formula being:

$${\hat{x}}^{( 1 )} ( k ) = [ {\sum }_{i = 1}^{n} {w}^{n - i} {x}^{( 1 )} ( i ) - \frac{b}{a} ] {e}^{- a ( k - t )} + \frac{b}{a} ;$$

where w^{n-k} denotes the target dynamic weight coefficient, \hat{x}⁽¹⁾(k) denotes the predicted cumulative number corresponding to the time period to be predicted, and x⁽¹⁾(i) denotes a historical cumulative number corresponding to the current historical time period.

Optionally, the coefficient determination unit is specifically used for performing computation on the historical cumulative number corresponding to each current historical time period using the PSO algorithm to obtain a two-dimensional vector [w,t]^{T}; and an initial dynamic weight coefficient is extracted from the two-dimensional vector, and the initial dynamic weight coefficient is taken as the target dynamic weight coefficient.

Optionally, the coefficient determination unit is specifically used for performing computation on the historical cumulative number corresponding to each current historical time period using the PSO algorithm to obtain the two-dimensional vector [w,t]^{T}; the initial dynamic weight coefficient is extracted from the two-dimensional vector; the initial dynamic weight coefficient is introduced as a constant into McLaughlin series; and the McLaughlin series is calculated using an ant colony optimization algorithm (ACO) algorithm to obtain a final dynamic weight coefficient, and the final dynamic weight coefficient is taken as the target dynamic weight coefficient.

Optionally, the coefficient determination unit includes an intermediate parameter calculation subunit, a determination unit and a coefficient determination subunit. The intermediate parameter calculation subunit is used for calculating an intermediate dynamic weight parameter using the ACO algorithm, where the number of calculation times is incremented by one with each calculation of the intermediate dynamic weight parameter. The determination unit is used for determining whether the number of current calculation times reaches a preset number of times. The coefficient determination subunit is used for taking an intermediate dynamic weight parameter when the number of current calculation times reaches the preset number of times as the target dynamic weight coefficient.

Optionally, the coefficient determination unit is further specifically used for determining a reference number corresponding to each current historical time period according to the target dynamic weight coefficient corresponding to each current historical time period and the historical cumulative number corresponding to each current historical time period; accumulating and substituting a product of the reference number corresponding to each current historical time period and a preset coefficient into a grey differential equation, where the grey differential equation includes a correspondence relation between the greyscale driving coefficient b and the development coefficient a; and solving the grey differential equation by a least square method to obtain the development coefficient a and the greyscale driving coefficient b.

Optionally, the historical confirmed number determination module 310 is specifically used for crawling a website to obtain a number of historical confirmed cases corresponding to each of a plurality of historical time periods adjacent to the time period to be predicted.

The device for predicting the number of confirmed cases of the infectious disease provided by the embodiment of the present disclosure can implement the method for predicting the number of confirmed cases of the infectious disease provided by any one of embodiments of the present disclosure and has corresponding functional modules and beneficial effects for executing the method. The content not described in detail in embodiments of the present disclosure may refer to descriptions in any method embodiment of the present disclosure.

### Embodiment Four

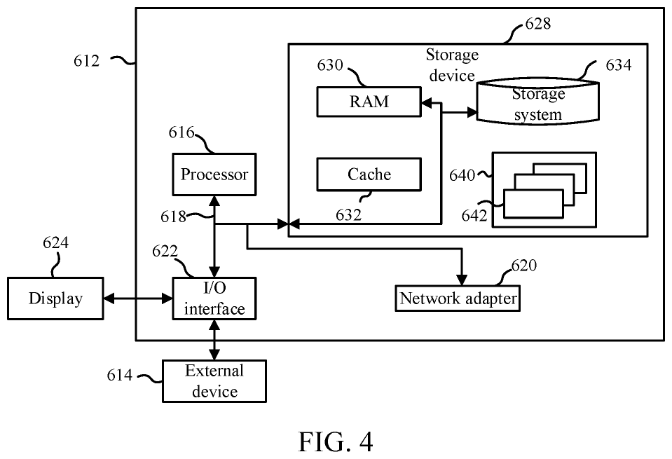

FIG. 4 is a block diagram of an apparatus according to Embodiment four of the present disclosure. FIG. 4 illustrates a block diagram of an exemplary apparatus 612 suitable for implementing embodiments of the present disclosure. The apparatus 612 illustrated in FIG. 4 is merely an example and should not impose any limitation on the function and scope of use of embodiments of the present disclosure.

As illustrated in FIG. 4, the apparatus 612 is represented in a form of a universal apparatus. Assemblies of the apparatus 612 may include, but are not limited to, one or more processors 616, a storage device 628, and a bus 618 connecting different system assemblies (include the storage device 628 and the processor 616).

The bus 618 represents one or more of several types of bus structures, and includes a storage device bus or a storage device controller, a peripheral bus, a graphics acceleration port, a processor, or a local bus using any of various bus structures. For example, these architectures include, but are not limited to, an industry subversive alliance (ISA) bus, a micro channel architecture (MAC) bus, an enhanced ISA bus, a video electronics standards association (VESA) local bus, and a peripheral component interconnect (PCI) bus.

The apparatus 612 typically includes a variety of computer system readable media. The computer system readable media may be any available medium that can be accessed by the apparatus 612, and include a volatile medium and nonvolatile medium, a removable medium and non-removable medium.

The storage device 628 may include a computer system readable medium in a form of volatile memory, such as random access memory (RAM) 630 and/or a cache memory 632. A terminal 612 may further include other removable/non-removable and volatile/non-volatile computer system storage media. Only as an example, a storage system 634 may be used to read and write non-removable and nonvolatile magnetic media (not shown in FIG. 4, generally referred to as a “hard disk drive”). Although FIG. 4 does not show a magnetic disk drive for providing reading and writing to a removable nonvolatile magnetic disk (such as a floppy disk) and an optical disk drive for providing reading and writing to the removable nonvolatile optical disk, such as a compact disc read-only memory (CD-ROM), a digital video disc-read only memory (DVD-ROM), or other optical media, in these cases, each driver may be connected to the bus 618 through one or more data medium interfaces. The storage device 628 may include at least one computer program product having a group (for example, at least one) of computer program modules, and the computer program modules are configured to execute the functions of embodiments of the present disclosure.

Computer programs/utilities 640 having the group (at least one) of computer program module 642 may be stored in, for example, the storage device 628. The computer program module 642 includes, but is not limited to, an operating system, one or more application computer programs, other computer program modules and computer program data, each or a certain combination of the examples may include an implementation of a network environment. The computer program module 642 generally executes functions and/or methods in the described embodiments of the present disclosure.

The apparatus 612 may also communicate with one or more external apparatuses 614 (such as a keyboard, a pointing terminal, a display 624 and the like), may further communicate with one or more terminals that enable a user to interact with the apparatus 612, and/or communicate with any terminal (such as a network card, a modem, and the like) that enables the apparatus 612 to communicate with one or more other computing terminals. The communication may be performed through an input/output (VO) interface 622. Furthermore, the apparatus 612 may further communicate with one or more networks (such as a local area network (LAN), a wide area network (WAN), and/or a public network, such as the Internet) through a network adapter 620. As shown in FIG. 4, the network adapter 620 communicates with other modules of the apparatus 612 through the bus 618. It should be understood that although not shown in the figure, other hardware and/or software modules may be used in conjunction with the apparatus 612, the hardware and/or software modules include, but are not limited to, microcode, terminal drives, redundant processors, external disk drive arrays, a redundant arrays of independent disk (RAID) system, tape drives, and data backup storage systems.

The processor 616 implements various functional applications and data processing by executing the computer program stored in the storage device 628, for example, implements the method for predicting the number of confirmed cases of the infectious disease provided by any embodiment of the present disclosure, and the method may include the following operations.

A number of historical confirmed cases corresponding to each of a plurality of historical time periods adjacent to a time period to be predicted is obtained.

A historical cumulative number corresponding to each current historical time period is determined according to a sum of a number of historical confirmed cases corresponding to a current historical time period and numbers of historical confirmed cases corresponding to previous target historical time periods.

A predicted cumulative number corresponding to the time period to be predicted is determined based on the historical cumulative number corresponding to each current historical time period.

A differential value between the predicted cumulative number corresponding to the time period to be predicted and a historical cumulative number corresponding to current target historical time period adjacent to the time period to be predicted is determined, and the differential value is taken as a predicted confirmed number of the time period to be predicted.

In the technical solution of the embodiments of the present disclosure, the number of historical confirmed cases corresponding to each of the plurality of historical time periods adjacent to the time period to be predicted is acquired; the historical cumulative number corresponding to each current historical time period is determined according to the sum of the number of historical confirmed cases corresponding to the current historical time period and the number of historical confirmed cases corresponding to the previous target historical time period; the predicted cumulative number corresponding to the time period to be predicted is determined based on the historical cumulative number corresponding to each current historical time period; and the differential value between the predicted cumulative number corresponding to the time period to be predicted and the historical cumulative number corresponding to current target historical time period adjacent to the time period to be predicted is determined, and the differential value is taken as the predicted confirmed number of the time period to be predicted. In the existing prediction which is predicted by the increasing number, the prediction result may be inaccurate when there is fluctuation in the increasing number. Compared with the existing prediction, this embodiment derives the rule of continuous growth by predicting based on the historical cumulative number, thereby ensuring the accuracy of the prediction, improving the accuracy of the predicting the number of future confirmed cases.

### Embodiment Five

Embodiment five of the present disclosure further provides a computer-readable storage medium configured to store computer programs. When executed by a processor, the computer programs implement the method for predicting a number of confirmed cases of an infectious disease of any one of embodiments of the present disclosure. The method may include the following operations.

A number of historical confirmed cases corresponding to each of a plurality of historical time periods adjacent to a time period to be predicted is obtained.

A historical cumulative number corresponding to each current historical time period is determined according to a sum of a number of historical confirmed cases corresponding to a current historical time period and numbers of historical confirmed cases corresponding to previous target historical time periods.

A predicted cumulative number corresponding to the time period to be predicted is determined based on the historical cumulative number corresponding to each current historical time period.

A differential value between the predicted cumulative number corresponding to the time period to be predicted and a historical cumulative number corresponding to current target historical time period adjacent to the time period to be predicted is determined, and the differential value is taken as a predicted confirmed number of the time period to be predicted.

The computer-readable storage medium of the embodiment of the present disclosure may adopt any combination of one or more computer-readable media. The computer-readable medium may be a computer-readable signal medium or a computer-readable storage medium. The computer-readable storage medium may be, for example, but not limited to, electrical, magnetic, optical, electromagnetic, infrared, or semi-conductive systems, devices or components, or any combination of the above. More specific examples (non-exhaustive list) about the computer-readable storage medium include: an electrical connection with one or more wires, a portable computer disk, a hard disk, a random access memory (RAM), a read only memory (ROM), an erasable programmable read only memory (EPROM or a flash memory), optical fiber, a portable compact disk read only memory (CD-ROM), an optical memory component, a magnetic memory component, or any suitable combination of the above. In this document, the computer-readable storage medium may be any tangible medium containing or storing computer programs, and the computer programs may be used by or in combination with an instruction execution system, device, or component.

The computer-readable signal medium may include a data signal propagating in a baseband or as part of a carrier wave, and the data signal carries computer-readable computer program codes. Such propagated data signals may take a variety of forms, including but not limited to electromagnetic signals, optical signals, or any suitable combination of the above. The computer-readable signal medium may also be any computer-readable medium other than the computer-readable storage medium, and the computer-readable medium may transmit, propagate, or transmit computer programs, where the computer programs are used by or in combination with the instruction execution system, the device, or the component.

The computer program codes contained on the storage medium may be transmitted by any suitable medium, including, but not limited to, wireless, a wire, a cable, radio frequency (RF), and the like, or any suitable combination of the above.

The computer program codes for executing operations of the present disclosure may be written in one or more computer program languages or combinations thereof. The computer programming languages include object-oriented computer programming languages such as Java, Smalltalk, C++, as well as conventional procedural computer programming languages such as C language or similar computer programming languages. The computer program codes may be executed entirely on a user's computer, partly on the user's computer, as a separate software package, partly on the user's computer and partly on a remote computer, or entirely on the remote computer or a terminal. In a case of the remote computer, the remote computer may be connected to the user's computer through any kind of network, including a local area network (LAN) or a wide area network (WAN), or may be connected to an external computer (for example, connected through the Internet by using an Internet service provider).

In the technical solution of the embodiments of the present disclosure, the number of historical confirmed cases corresponding to each of the plurality of historical time periods adjacent to the time period to be predicted is acquired; the historical cumulative number corresponding to each current historical time period is determined according to the sum of the number of historical confirmed cases corresponding to the current historical time period and the number of historical confirmed cases corresponding to the previous target historical time period; the predicted cumulative number corresponding to the time period to be predicted is determined based on the historical cumulative number corresponding to each current historical time period; and the differential value between the predicted cumulative number corresponding to the time period to be predicted and the historical cumulative number corresponding to current target historical time period adjacent to the time period to be predicted is determined, and the differential value is taken as the predicted confirmed number of the time period to be predicted. In the existing prediction which is predicted by the increasing number, the prediction result may be inaccurate when there is fluctuation in the increasing number. Compared with the existing prediction, this embodiment derives the rule of continuous growth by predicting based on the historical cumulative number, thereby ensuring the accuracy of the prediction, improving the accuracy of predicting the number of future confirmed cases.

The foregoing merely depicts some illustrative embodiments of the present disclosure and the technical principles used herein. Those skilled in the art will appreciate that the present disclosure will not be limited to the specific embodiments described herein. Those skilled in the art will be able to make various apparent modifications, adaptations and substitutions without departing from the scope of the present disclosure. Therefore, while the present disclosure has been described in detail through the foregoing embodiments, the present disclosure will not be limited to the above-mentioned embodiments and may further include many other equivalent embodiments without departing from the concept of the present disclosure. The scope of the present disclosure is thus only determined in and by the appended claims.

1. A method for predicting a number of confirmed cases of an infectious disease, comprising:
obtaining a number of historical confirmed cases corresponding to each of a plurality of historical time periods adjacent to a time period to be predicted;
summing up a number of historical confirmed cases corresponding to a current historical time period with numbers of historical confirmed cases corresponding to previous target historical time periods, to determine a historical cumulative number corresponding to each current historical time period;
determining a predicted cumulative number corresponding to the time period to be predicted based on the historical cumulative number corresponding to each current historical time period; and
determining a differential value between the predicted cumulative number corresponding to the time period to be predicted and the historical cumulative number corresponding to the current target historical time period adjacent to the time period to be predicted, and taking the differential value as a predicted confirmed number of the time period to be predicted.

2. The method of claim 1, wherein determining the predicted cumulative number corresponding to the time period to be predicted based on the historical cumulative number corresponding to each current historical time period comprises:
performing computation on the historical cumulative number corresponding to each current historical time period using a particle swarm optimization (PSO) algorithm, to determine a target dynamic weight coefficient corresponding to each current historical time period;
determining a development coefficient a and a greyscale driving coefficient b based on the historical cumulative number corresponding to each current historical time period; and
determining the predicted cumulative number corresponding to the time period to be predicted based on a first preset formula, is the first preset formula being:
$${\hat{x}}^{( 1 )} ( k ) = [ {\sum }_{i = 1}^{n} {w}^{n - i} {x}^{( 1 )} ( i ) - \frac{b}{a} ] {e}^{- a ( k - t )} + \frac{b}{a} ;$$
where w^{n-k} denotes the target dynamic weight coefficient, \hat{x}⁽¹⁾(k) denotes the predicted cumulative number corresponding to the time period to be predicted, and x⁽¹⁾(i) denotes the historical cumulative number corresponding to the current historical time period.

3. The method of claim 2, wherein performing computation on the historical cumulative number corresponding to each current historical time period using the PSO algorithm to determine the target dynamic weight coefficient corresponding to each current historical time period comprises:
performing computation on the historical cumulative number corresponding to each current historical time period using the PSO algorithm to obtain a two-dimensional vector [w,t]^{T}; and
extracting an initial dynamic weight coefficient from the two-dimensional vector, and using the initial dynamic weight coefficient as the target dynamic weight coefficient.

4. The method of claim 2, wherein performing computation on the historical cumulative number corresponding to each current historical time period using the PSO algorithm to determine the target dynamic weight coefficient corresponding to each current historical time period comprises:
performing computation on the historical cumulative number corresponding to each current historical time period using the PSO algorithm to obtain a two-dimensional vector [w,t]^{T};
extracting an initial dynamic weight coefficient from the two-dimensional vector;
introducing the initial dynamic weight coefficient as a constant into McLaughlin series; and
performing computation on the McLaughlin series using an ant colony optimization algorithm (ACO) algorithm to obtain a final dynamic weight coefficient, and using the final dynamic weight coefficient as the target dynamic weight coefficient.

5. The method of claim 4, wherein performing computation on the McLaughlin series using the ACO algorithm to obtain the final dynamic weight coefficient and using the final dynamic weight coefficient as the target dynamic weight coefficient comprises:
calculating an intermediate dynamic weight parameter using the ACO algorithm, wherein the number of calculation times is incremented by one with each calculation of the intermediate dynamic weight parameter;
determining whether the number of current calculation times reaches a preset number of times; and
taking the corresponding intermediate dynamic weight parameter when the number of current calculation times reaches the preset number of times as the target dynamic weight coefficient.

6. The method of claim 2, wherein determining the development coefficient a and the greyscale driving coefficient b based on the historical cumulative number corresponding to each current historical time period comprises:
determining a reference number corresponding to each current historical time period based on the target dynamic weight coefficient corresponding to each current historical time period and the historical cumulative number corresponding to the current historical time period;
accumulating the product of the reference number corresponding to each current historical time period with a preset coefficient and substituting the result into a grey differential equation, wherein the grey differential equation comprises a correspondence relation between the greyscale driving coefficient b and the development coefficient a; and
solving the grey differential equation by a least square method to obtain the development coefficient a and the greyscale driving coefficient b.

7. The method of claim 1, wherein obtaining the number of historical confirmed cases corresponding to each of the plurality of historical time periods adjacent to the time period to be predicted comprises:
crawling a website to obtain the number of historical confirmed cases corresponding to each of the plurality of historical time periods adjacent to the time period to be predicted.

8. A device for predicting a number of confirmed cases of an infectious disease, comprising:
a historical confirmed number determination module, configured for obtaining a number of historical confirmed cases corresponding to each of a plurality of historical time periods adjacent to a time period to be predicted;
a historical cumulative number determination module, configured for summing up a number of historical confirmed cases corresponding to a current historical time period with numbers of historical confirmed cases corresponding to previous target historical time periods, to determine a historical cumulative number corresponding to each current historical time period;
a predicted cumulative number determination module, configured for determining a predicted cumulative number corresponding to the time period to be predicted based on the historical cumulative number corresponding to each current historical time period; and
a predicted confirmed number determination module, configured for determining a differential value between the predicted cumulative number corresponding to the time period to be predicted and the historical cumulative number corresponding to current target historical time period adjacent to the time period to be predicted, and taking the differential value as a predicted confirmed number of the time period to be predicted.

9. The device of claim 8, wherein predicted cumulative number determination module comprises:
a coefficient determination unit, configured for performing computation on the historical cumulative number corresponding to each current historical time period using a particle swarm optimization (PSO) algorithm, to determine a target dynamic weight coefficient corresponding to each current historical time period, and determining a development coefficient a and a greyscale driving coefficient b based on the historical cumulative number corresponding to each current historical time period; and
a predicted cumulative number determination unit, configured for determining the predicted cumulative number corresponding to the time period to be predicted based on a first preset formula, is the first preset formula being:
$${\hat{x}}^{( 1 )} ( k ) = [ {\sum }_{i = 1}^{n} {w}^{n - i} {x}^{( 1 )} ( i ) - \frac{b}{a} ] {e}^{- a ( k - t )} + \frac{b}{a} ;$$
where w^{n-k} denotes the target dynamic weight coefficient, \hat{x}⁽¹⁾(k) denotes the predicted cumulative number corresponding to the time period to be predicted, and x⁽¹⁾(i) denotes the historical cumulative number corresponding to the current historical time period.

10. The device of claim 9, wherein the coefficient determination unit is configured for performing computation on the historical cumulative number corresponding to each current historical time period using the PSO algorithm to obtain a two-dimensional vector [w,t]^{T}; and
extracting an initial dynamic weight coefficient from the two-dimensional vector, and using the initial dynamic weight coefficient as the target dynamic weight coefficient.

11. The device of claim 9, wherein the coefficient determination unit is configured for performing computation on the historical cumulative number corresponding to each current historical time period using the PSO algorithm to obtain a two-dimensional vector [w,t]^{T};
extracting an initial dynamic weight coefficient from the two-dimensional vector;
introducing the initial dynamic weight coefficient as a constant into McLaughlin series; and
performing computation on the McLaughlin series using an ant colony optimization algorithm (ACO) algorithm to obtain a final dynamic weight coefficient, and using the final dynamic weight coefficient as the target dynamic weight coefficient.

12. The device of claim 11, wherein the coefficient determination unit comprises:
an intermediate parameter calculation subunit, configured for calculating an intermediate dynamic weight parameter using the ACO algorithm, wherein the number of calculation times is incremented by one with each calculation of the intermediate dynamic weight parameter;
a determination unit, configured for determining whether the number of current calculation times reaches a preset number of times; and
a coefficient determination subunit, configured for taking the corresponding intermediate dynamic weight parameter when the number of current calculation times reaches the preset number of times as the target dynamic weight coefficient.

13. The device of claim 9, wherein the coefficient determination unit is configured for determining a reference number corresponding to each current historical time period based on the target dynamic weight coefficient corresponding to each current historical time period and the historical cumulative number corresponding to the current historical time period;
accumulating the product of the reference number corresponding to each current historical time period with a preset coefficient and substituting the result into a grey differential equation, wherein the grey differential equation comprises a correspondence relation between the greyscale driving coefficient b and the development coefficient a; and
solving the grey differential equation by a least square method to obtain the development coefficient a and the greyscale driving coefficient b.

14. The device of claim 8, wherein the historical confirmed number determination module is configured for crawling a website to obtain the number of historical confirmed cases corresponding to each of the plurality of historical time periods adjacent to the time period to be predicted.

15. An apparatus, comprising:
one or more processors;
a storage device storing one or more computer programs, which when executed by the one or more processors cause the one or more processors to perform the method for predicting a number of confirmed cases of an infectious disease as recited in claim 1.

16. The apparatus of claim 15, wherein determining the predicted cumulative number corresponding to the time period to be predicted based on the historical cumulative number corresponding to each current historical time period comprises:
performing computation on the historical cumulative number corresponding to each current historical time period using a particle swarm optimization (PSO) algorithm, to determine a target dynamic weight coefficient corresponding to each current historical time period;
determining a development coefficient a and a greyscale driving coefficient b based on the historical cumulative number corresponding to each current historical time period; and
determining the predicted cumulative number corresponding to the time period to be predicted based on a first preset formula, is the first preset formula being:
$${\hat{x}}^{( 1 )} ( k ) = [ {\sum }_{i = 1}^{n} {w}^{n - i} {x}^{( 1 )} ( i ) - \frac{b}{a} ] {e}^{- a ( k - t )} + \frac{b}{a} ;$$
where w^{n-k} denotes the target dynamic weight coefficient, \hat{x}⁽¹⁾(k) denotes the predicted cumulative number corresponding to the time period to be predicted, and x⁽¹⁾(i) denotes the historical cumulative number corresponding to the current historical time period.

17. The apparatus of claim 16, wherein performing computation on the historical cumulative number corresponding to each current historical time period using the PSO algorithm to determine the target dynamic weight coefficient corresponding to each current historical time period comprises:
performing computation on the historical cumulative number corresponding to each current historical time period using the PSO algorithm to obtain a two-dimensional vector [w,t]^{T}; and
extracting an initial dynamic weight coefficient from the two-dimensional vector, and using the initial dynamic weight coefficient as the target dynamic weight coefficient.

18. A computer-readable storage medium, storing a computer program, which when executed by a processor causes the processor to perform the method for predicting a number of confirmed cases of an infectious disease as recited in claim 1.

19. The computer-readable storage medium of claim 18, wherein determining the predicted cumulative number corresponding to the time period to be predicted based on the historical cumulative number corresponding to each current historical time period comprises:
performing computation on the historical cumulative number corresponding to each current historical time period using a particle swarm optimization (PSO) algorithm, to determine a target dynamic weight coefficient corresponding to each current historical time period;
determining a development coefficient a and a greyscale driving coefficient b based on the historical cumulative number corresponding to each current historical time period; and
determining the predicted cumulative number corresponding to the time period to be predicted based on a first preset formula, is the first preset formula being:
$${\hat{x}}^{( 1 )} ( k ) = [ {\sum }_{i = 1}^{n} {w}^{n - i} {x}^{( 1 )} ( i ) - \frac{b}{a} ] {e}^{- a ( k - t )} + \frac{b}{a} ;$$
where w^{n-k} denotes the target dynamic weight coefficient, \hat{x}⁽¹⁾(k) denotes the predicted cumulative number corresponding to the time period to be predicted, and x⁽¹⁾(i) denotes the historical cumulative number corresponding to the current historical time period.

20. The computer-readable storage medium of claim 19, wherein performing computation on the historical cumulative number corresponding to each current historical time period using the PSO algorithm to determine the target dynamic weight coefficient corresponding to each current historical time period comprises: performing computation on the historical cumulative number corresponding to each current historical time period using the PSO algorithm to obtain a two-dimensional vector [w,t]^{T}; and
extracting an initial dynamic weight coefficient from the two-dimensional vector, and using the initial dynamic weight coefficient as the target dynamic weight coefficient.
